(19) United States
(12) Patent Application Publication
(10) Pub. No.: US 2011/0144407 A1
(43) Pub. Date: Jun. 16, 2011

(54) PROCESS FOR PRODUCING PURIFIED HYDROCARBON HAS

(76) Inventors: Adriaan Pieter Houtekamer, Norfolk (GB); Paul Jason Williams, Richmond, TX (US)

(21) Appl. No.: 12/995,414

(22) PCT Filed: May 28, 2009
(86) PCT No.: PCT/EP09/56542

§ 371 Date: Feb. 28, 2011

Related U.S. Application Data
(60) Provisional application No. 61/057,356, filed on May 30, 2008.

(51) Int. Cl. C07C 7/144; C01B 3/32

(52) U.S. Cl. 585/818; 48/127.3

(57) ABSTRACT

Process for producing purified hydrocarbon gas from a gas stream comprising hydrocarbons and acidic contaminants, which process comprises the steps of: (a) contacting the gas stream with one or more membranes to obtain a hydrocarbon-rich retentate and a acidic contaminant-rich permeate; (b) cooling the hydrocarbon-rich retentate in a cooling stage to form a mixture comprising solid and/or liquid acidic contaminants and a vapour comprising vaporous hydrocarbons; (c) separating solid and/or liquid acidic contaminants from the mixture, yielding the purified hydrocarbon gas.

The invention relates to a process for producing purified hydrocarbon gas. The invention especially relates to a process for producing purified hydrocarbon gas from hydrocarbon gas that contains hydrocarbons and acidic contaminants such as carbon dioxide and hydrogen sulphide.

Such a process is known from WO-A 2004/070297. This document discloses a process in which a natural gas stream, comprising hydrocarbons and acidic contaminants, is first cooled to remove water from the natural gas, and subsequently the natural gas is further cooled to solidify acidic contaminants or dissolve such contaminants in a liquid, which contaminants are removed so that a purified natural gas is recovered. WO-A 2004/070297 is herein incorporated by reference in its entirety.

It has been found that this process is very suitable when the natural gas stream contains relatively small amounts of acidic contaminants, such as up to 25% vol. However, there is room for improvement of this process when the natural gas streams contain high concentrations, i.e. at least 25% vol, of acidic contaminants.

A two-step process is known from WO-A 2007/030888, which discloses a process in which a natural gas stream comprising hydrocarbons and acidic species is dehydrated and subsequently cooled by adiabatic expansion to obtain a slurry of solid acidic contaminants and liquid hydrocarbons together with a gaseous stream containing gaseous acidic species. The slurry is removed and the gaseous stream containing the gaseous acidic species is treated with a solvent, e.g., methanol, to wash the gaseous acidic species from the gaseous stream, resulting in a purified natural gas product. The acidic species are contained in the solvent, and are recovered from the solvent in a subsequent desorption step. The solvent may be recycled to the wash treatment after a number of heat exchange steps. WO-A 2007/030888 is herein incorporated by reference in its entirety.

This process requires a cumbersome recovery of the solvent in a desorption step and it also requires heat exchange steps before recycling the solvent to the wash treatment. Moreover, the wash treatment is conducted at a significantly lower pressure than the cooling and removal steps, which has an effect on the size and the costs of the equipment for such wash treatment.

Another known method of purifying natural gas streams is by using a gas permeable membrane. Such a process is e.g. known from U.S. Pat. No. 5,411,721. This method employs a gas permeable membrane to separate the majority of CO₂ from a natural gas stream in the permeate. The permeate also contains significant amounts of hydrocarbons and therefore, the permeate is treated in a pressure-swing absorption unit to recover the hydrocarbons. It is evident that this method also requires absorption and desorption steps similar to those in the process of WO-A 2007/030888. A similar two-stage process has been described in U.S. Pat. No. 5,407,466, wherein a natural gas stream is subjected to separation of hydrogen sulphide, carbon dioxide and water by means of two different membranes followed by an absorption process. In cases wherein two membrane units are used, the process still also utilises a further absorption step for acid gas removal. U.S. Pat. No. 5,411,721 is herein incorporated by reference in its entirety.

In US-A 2007/0272079 a process is shown in which three membrane units are used. The process also requires a compression step between two units. Further, it teaches a recirculation of the residue stream of the second membrane unit to the first membrane unit. These additional steps add to the complexity and costs of the process. US-A 2007/0272079 is herein incorporated by reference in its entirety.

There is a need in the art for an efficient method for removal of acidic contaminants from gases such as natural gas with a high content of acidic contaminants can be obtained without the need for a complex wash unit or expensive adsorption/desorption steps.

Accordingly, the present invention provides a process for producing purified hydrocarbon gas from a gas stream comprising hydrocarbons and acidic contaminants, which process comprises the steps of:

(a) contacting the gas stream with one or more membranes to obtain a hydrocarbon-rich retentate and a acidic contaminant-rich permeate;
(b) cooling the hydrocarbon-rich retentate in a cooling stage to form a mixture comprising solid and/or liquid acidic contaminants and a vapour comprising vaporous hydrocarbons;
(c) separating solid and/or liquid acidic contaminants from the mixture, yielding the purified hydrocarbon gas

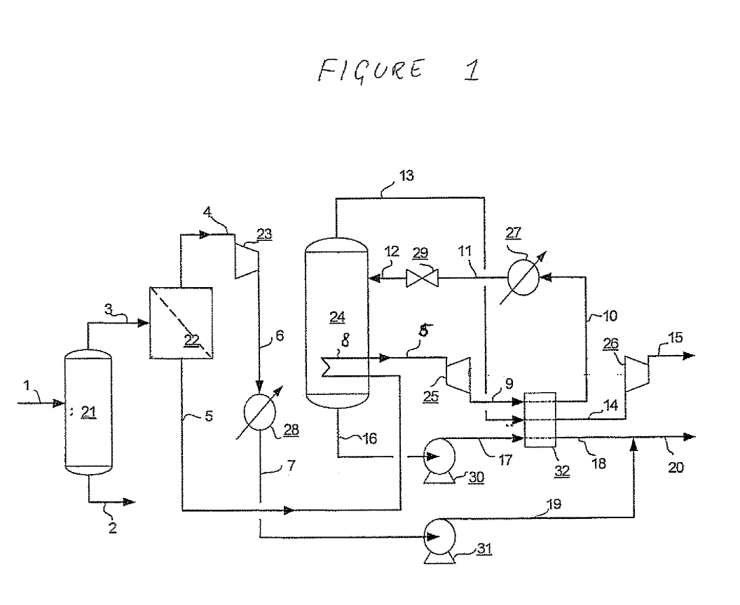

FIG. 1 shows a flow scheme of a system to treat a gas stream according to one embodiment of the invention.

The present process provides a solution to the purification of gas streams that contain relatively large amounts of acidic contaminants. In the first contacting stage a large proportion of the acidic contaminants are separated and subsequently removed, whereas the hydrocarbon-rich retentate contains the gaseous hydrocarbons and a reduced amount of vaporous acidic contaminants. This step can be conducted at the prevailing pressure of the gas stream, which has benefits vis-à-vis the size and costs of the one or more membrane units. Further, the hydrocarbon-rich retentate becomes available for the cooling step at substantially the same pressure as the one prevailing in the membrane-contacting step. This allows for efficient cooling of the hydrocarbon-rich retentate by expansion. Moreover, the amount of gas that is to be cooled, e.g., by expansion, has been reduced which also has a beneficial effect on the size, complexity and costs of the cooling unit.

The gas stream can be any stream of gas that comprises acidic contaminants and hydrocarbons. In particular the process according to the present invention can be applied to a natural gas stream, i.e., a gas stream that contains significant amounts of hydrocarbons, in particular methane, and that has been produced from a subsurface reservoir. It includes a methane natural gas stream, an associated gas stream or a coal bed methane stream. The amount of the hydrocarbon fraction in such a gas stream is suitably from 10 to 85 mol % of the gas stream, preferably from 20 to 75 mol %. Especially the hydrocarbon fraction of the gas stream comprises at least 75 mol % of methane, preferably at least 90 mol %. The hydrocarbon fraction in the natural gas stream suitably contains from 0 to 25 mol %, suitably from 0.1 to 10 mol %, of C₂-C₆ compounds. The gas stream may also comprise up to 20 mol %, suitably from 0.1 to 10 mol % of nitrogen, based on total gas stream.

In the process of the invention the gas stream comprises suitably hydrogen sulphide and/or carbon dioxide as acidic contaminants. It is observed that also minor amounts of other contaminants may be present, e.g. carbon oxysulphide, mercaptans, alkyl sulphides and aromatic sulphur-containing compounds. The major part of these components will also be removed in the process of the present invention.

The amount of hydrogen sulphide in the gas stream containing methane is suitably in the range of from 5 to 40 volume % of the gas stream, preferably from 20 to 35 volume % and/or the amount of carbon dioxide is in the range of from 10 to 90 vol %, preferably from 20 to 75 vol %, based on the total gas stream. It is observed that the present process is especially suitable for gas streams comprising large amounts of contaminants, e.g. 10 vol % or more, suitably between 15 and 90 vol %.

Gas stream containing the large amounts of contaminants as described above cannot be processed using conventional techniques as amine extraction techniques as they will become extremely expensive, especially due to the large amounts of heat needed for the regeneration of loaded amine solvent.

Gas streams such as natural gas streams may become available at a temperature of −5 to 150° C. Since the one or more membranes do not require that the gas is at a specifically low temperature the membrane separation step may be conducted at the temperature at which the gas becomes available, and there is no need for heat exchange. This represents a significant advantage. Natural gas may also become available at a pressure of 10 to 700 bar, suitably from 20 to 200 bar. It is an advantage of the present invention that the first separation step can be conducted at the pressure at which the gas becomes available without affecting the membrane separation performance. Although it is possible to expand the natural gas stream or compress it, it is preferred to contact the natural gas stream with the membrane at the pressure at which it becomes available.

The first contacting step may be conducted in one or more membrane units. If more than one unit is being used, they may be used in a parallel or sequential manner. If they are used in a parallel manner, all retentate streams and all permeate streams are preferably combined to form one combined retentate stream and one combined permeate stream, and the combined streams are processed further. Alternatively, the units may be in series. In such cases either of the permeate or retentate streams of a membrane unit may be passed to a subsequent membrane unit. Suitably, the permeate stream from a membrane unit is subjected to contact with a subsequent membrane unit to yield a subsequent permeate and a subsequent retentate. The retentate of any subsequent membrane unit may be combined with the retentate of previous unit or units. It is also possible to recycle a retentate from a subsequent membrane unit to the original gas stream that is fed to the first membrane unit. Alternatively, it may be suitable that a permeate is compressed, e.g., to a pressure from 30 to 220 bar, and be recycled to the original gas stream. The number of membrane units will vary depending on feed composition.

The operation parameters of a membrane unit or units to separate acidic contaminants from gas are known in the art. Such operation parameters include the process conditions under which the natural gas stream is contacted with the membrane and also the type of membrane used. As indicated above, it is advantageous that the present invention allows the separation step over a membrane to be conducted under process conditions that are substantially equal to the conditions at which the gas stream becomes available. Suitably, the gas stream is contacted with the membrane at a temperature ranging from −5 to 150° C. and a pressure ranging from 20 to 200 bar.

In order to ensure that all acidic components in the gas stream remain vaporous so that they can easily permeate through the membrane, it is preferred that the gas stream is contacted with the membrane at a temperature of at least 1° C. above the dew point of the gas stream. Such could be at least 1° C. above the boiling point of the first of the acidic contaminants to condense.

Membranes to be used in the process of the present invention are known in the art. It is advantageous to use membranes with a high selectivity for acidic contaminants as carbon dioxide and hydrogen sulphide. The selectivity is defined as the ratio of the acidic contaminants permeance over the permeance of the hydrocarbons in the mixture. Preferably, the selectivity of the membrane in the contacting step is from 10 to 200, preferably from 20 to 150.

The permeance for carbon dioxide or hydrogen sulphide of the membrane in the contacting step is suitably from 10⁻⁴° to 10⁻⁴ mol/m²sPa, preferably from 10⁻⁹ to 10⁻⁵ mol/m²sPa. If more than one membrane unit is used, the membranes may be the same or different.

As to the type of membrane used, the skilled person may easily choose from a variety of membranes. In accordance with the teachings of U.S. Pat. No. 5,407,466 one may want to use polysulphides or polysulphone material or, in particular, cellulose acetate or cellulose triacetate. The process of US-A 2007/0272079 is suitably carried out using fluorinated dioxoles or dioxolanes or polyimides. Since molecular sieve type of membranes have a very good separation selectivity, it is preferred that the membrane used comprises a molecular sieve membrane. Molecular sieves can be chosen from a variety of crystalline materials including zeolites, AlPOs, SAPOs (silicoaluminophosphate), and other materials such as molecular sieve carbon and silica. Molecular sieve membranes can be free-standing or supported on a porous substrate, such as a porous tube. The use of SAPO-34 molecular sieve is especially preferred. A suitable process for the manufacture of such SAPO-34 membranes has been described in U.S. Pat. No. 7,316,727.

The contaminant-rich permeate may be removed from the process, optionally after further treatment, e.g. the recovery of any hydrocarbons that have been entrained in the permeate. It is also possible to combine the permeate with acidic contaminants from other process streams. It may be advantageous to compress and/or condense the permeate stream and prepare it for underground injection, e.g., for enhanced recovery of oil and/or gas.

After separation, the hydrocarbon-rich retentate is cooled. The cooling may be effected by any known method, such as indirect heat exchange and expansion. Alternatively, a direct heat exchange, e.g., by spraying with a cold liquid, as shown in WO-A 2004/070297, is also possible. The skilled person will appreciate that expansion, suitably adiabatic expansion, causes a lowering of temperature, so that cooling may be achieved by expansion and adapting the pressure. The cooling may be conducted in several steps. It is preferred that the hydrocarbon-rich retentate is subjected to heat exchange with one or more other cold process streams or external streams. Cold external streams may suitably be streams from an LNG (liquefied natural gas) plant, such as a cold LNG stream or a refrigerant stream, or from an air separation unit. One suitable process stream comprises the solid and/or liquid acidic contaminants obtained in the cooling step. Preferably, the cooling stage of the hydrocarbon-rich retentate comprises one or more expansion steps. For this purpose conventional equipment may be used. Conventional equipment includes turbo-expanders and so-called Joule-Thomson valves or venturi tubes. It is preferred to at least partly cool the hydrocarbon-rich retentate over a turbo-expander, releasing energy. One advantageous effect of using the turbo-expander is that the energy that is released in the turbo-expander can suitably be used, e.g., for compressing at least part of the purified hydrocarbon gas. Since the stream of the purified hydrocarbon gas is smaller than the stream of hydrocarbon-rich retentate now that acidic contaminants have been removed, the energy is suitably such that the purified hydrocarbon gas may be compressed to an elevated pressure that makes it suitable for transport in a pipeline.

The cooling steps eventually lead to the desired temperature at which acidic contaminants liquefy and/or solidify. However, since the hydrocarbon-rich retentate also may comprise hydrocarbons other than methane it is preferred to cool the hydrocarbon-rich retentate, suitably by expansion, to a temperature below the dew point of propane. In this way the vaporous hydrocarbon-rich retentate will develop liquid hydrocarbons, including propane, which can subsequently be recovered easily from the vapour.

As indicated above, the cooling eventually leads to solid and/or liquid acidic contaminants. It is preferred to achieve the cooling in several steps, e.g., by indirect heat exchange and/or expansion. It is also possible to solidify and/or liquefy by spraying with a cold liquid, as shown in WO-A 2004/070297. Suitably, solid and/or liquid acidic contaminants are obtained in a final expansion step. The final expansion step is preferably achieved over a Joule-Thomson valve. Therefore, preferably, in a first step, which may be achieved by various intermediate steps and various methods, the hydrocarbon-rich retentate is cooled to a temperature ranging from 1 to 40° C. above the freeze out temperature of the first acidic contaminant to freeze out, the freeze out temperature being the temperature at which solid contaminants are formed. Preferably, the cooling is effected till from 2 to 10° C. above the freeze out temperature. In a final step the hydrocarbon-rich retentate is preferably cooled to the temperature at which a mixture of solid and/or liquid acidic contaminants and a vapour comprising vaporous hydrocarbons are formed by expansion over a valve. Preferably, the hydrocarbon-rich retentate is partly or completely liquid before being expanded over the valve, and solid contaminants are formed upon expansion. This ensures a better separation performance. Suitably, the hydrocarbon-rich retentate is expanded from a pressure ranging from 40 to 200 bar to a pressure of 10 to 40 bar. Expansion over this pressure range suitably causes liquid and/or solid acidic contaminants to form. Preferably the expansion is done by isenthalpic expansion, preferably isenthalpic expansion over an orifice or a valve, especially a Joule-Thomson valve or a series of Joule-Thomson valves. In another preferred embodiment the expansion is done by nearly isentropic expansion, especially by means of an expander, preferably a turbo expander, or a laval nozzle.

Acidic contaminants that are usually present in gas streams, such as natural gas, include hydrogen sulphide and carbon dioxide. It is also possible that the natural gas stream contains other components, including ethane, propane and hydrocarbons with four or more carbon atoms. It will be appreciated that when a portion of acidic contaminants, e.g., carbon dioxide, liquefies and/or solidifies in the cooling stages, other components, e.g., hydrogen sulphide and hydrocarbons other than methane, may liquefy. The liquid components are suitably removed together with the solid and/or liquid acidic contaminants from the vapour.

The liquefaction and/or solidification of acidic contaminants may take place very rapidly especially upon expansion over a valve, thereby forming the mixture comprising solid and/or liquid acidic contaminants and a vapour comprising vaporous hydrocarbons. To facilitate the separation the mixture is passed into a vessel, wherein the separation between solid and/or liquid acidic contaminants and vapour can take place. By gravity the solid and/or liquid acidic contaminants, and any other liquid that is formed, drops to the bottom of the vessel. After such separation the solid acidic contaminants are removed from the process. Since it is easier to transport liquids than to transport solids, it is preferred to melt at least partly the solid acidic contaminants, if formed.

Such melting can be accomplished by heating the solids in the vessel by means of an electric immersion heater, by a bundle coil, i.e. a type of indirect heat exchanger, through which a process stream is fed, or by injecting a relatively warm fluid, such as a condensate. These measures have been suggested in WO-A 2004/0702897 and WO-A 2007/030888. In the present process it is preferred to heat at least a part of the withdrawn contaminants in a liquid, solid or slurry phase, and recycle at least a part of thus heated contaminants, in liquid or gaseous phase, to the vessel. In this way no extraneous material is recycled to the vessel. Preferably, at least 90%, more preferably at least 95% and most preferably at least 98% of the solid acidic contaminants are melted. In this way a liquid stream of contaminants is obtained, which can be easily transported further.

In a preferred embodiment, step (c) is performed in a separation vessel and is done using the steps of:

(c1) introducing a stream comprising liquid phase acidic contaminants into the intermediate or the bottom part or both of the separation vessel to obtain a diluted slurry of acidic contaminants;
(c2) introducing the diluted slurry of acidic contaminants via an eductor into a heat exchanger in which solid acidic contaminant present in the diluted slurry of contaminants is melted into liquid acidic contaminant, wherein the heat exchanger is positioned outside the separation vessel, and eductor is arranged inside or outside the separation device or partly inside and outside the separation vessel;
(c3) introducing part or all of the liquid contaminant obtained in step c2 into a gas-liquid separator, wherein the gas-liquid separator is preferably the bottom part of the separation vessel;
(c4) introducing part or all of the liquid contaminant obtained in step c3 into the separation vessel as described above;
(c5) removing from the gas-liquid separator a stream of liquid acidic contaminant and
(c6) separating the stream of liquid contaminant obtained in step c5 into a liquid product stream and a recirculation stream which is used as a motive fluid in the eductor.

In accordance with the present invention use is made of an eductor for removing the diluted slurry of contaminants from the separation device and passing/introducing said slurry into the heat exchanger. The diluted slurry of contaminants functions as the suction fluid in the eductor, whereas the recirculation stream to be introduced in the eductor in step c6 functions as the motive fluid.

Eductors, also referred to as siphons, exhausters, ejectors or jet pumps, are as such well-known and have extensively been described in the prior art. Reference herein to an eductor is to a device to pump produced solid and liquid CO2 slurry from the separator to the heat exchanger. The eductor is suitably designed for use in operations in which the head pumped against is low and is less than the head of the fluid used for pumping. Suitably, the eductor is a liquid jet pump. For a description of suitable eductors, also referred to as ejectors or jet pumps, reference is made to Perry's Handbook for Chemical Engineering, 8^{th} edition, chapter 10.2. In accordance with the present invention any type of eductor can be used. Also a configuration may be used in which multiple eductors are uses.

Preferably, the eductor is arranged inside the separation device or partly inside and outside the separation device.

Suitably, a housing can be positioned around the eductor, enabling the eductor to be removed from the separation device. Such a housing can, for instance, be a vessel like containment, e.g. a pipe, that can be isolated from the process through valves.

In another embodiment of the present invention the eductor is arranged outside the separation device. Such an embodiment can be useful in situations in which the eductor in use needs to be repaired or replaced.

The eductor can be of such a size that it fits completely in the separation device or it may cover the entire diameter of the separation device, usually a vessel. However, it may also extend at two locations through the internal wall of the separation device.

Suitably, between 0 and 90 vol % of the liquid phase contaminant obtained in step c2 is introduced in the separation device as described in step c1, preferably between 5 and 80 vol % of the liquid phase contaminant obtained in step c2. It is also possible to introduce all liquid phase contaminant obtained in step c2 in the separation device as described in step c1.

Suitably, between 10 and 100 vol % of the liquid phase contaminant obtained in step c2 is introduced into the bottom part of the separation device in step 8), preferably between 20 and 95 vol % of the liquid phase contaminant obtained in step c2. Suitably the remaining part is introduced into the intermediate part of the separation device.

In accordance with the present invention the heat exchanger is preferably arranged at a level positioned below the level at which the eductor is arranged.

Suitably, in step c6 between 25 and 95 vol % of the stream of liquid phase contaminant removed from the separation device in step c5 is used as a motive fluid in the eductor, preferably between 30 and 85 vol % of the stream of liquid phase contaminant removed from the separation device in step c5.

In step c3 of the process the liquid phase contaminant is preferably introduced into the bottom part of the separation device at a level which is higher than the level at which the liquid phase contaminant is removed from the bottom part of the separation device in step b). As a result free flash gas and/or vapor can escape to the top part of the cryogenic separation device.

Preferably, the eductor is arranged at a level which is higher than the level at which the heat exchanger is arranged, allowing the diluted slurry of contaminants to flow downstream into the heat exchanger.

It will be understood that the eductor is arranged below the slurry level which is maintained in the separation vessel.

The stream of liquid phase contaminant stream that is removed from the bottom part of the separation device is suitably removed at a level below the slurry level inside the separation device.

Suitable internals may be used to prevent ingress of solid particles into the withdrawal line. Preferably a pump is installed in the withdrawal line to remove the stream of liquid phase contaminant from the bottom part of the separation device, and to power the stream of liquid phase contaminant that is to be used as the motive fluid in the eductor.

In step c1 the recycle of the liquid phase contaminant can be introduced into the separation device and into the slurry of contaminants at a level lower than that at which the methane enriched gaseous phase is removed from the separation device. In this way a washing stream can be created over the inside walls of the device.

Preferably, means are positioned in the separation device to direct the diluted slurry of contaminants towards the eductor. Preferably, use is made of a funnel to establish this. One or more funnels can be arranged on top of each other. Preferably in the wider part of the funnel, a grid is present to stop large chunks of falling in the more narrow inlet of the eductor and in doing so, avoid plugging of the eductor.

In step c3 the liquid phase contaminant is preferably introduced into the bottom part of the separation device at a level which is higher than the level at which liquid phase contaminant is removed from the bottom part of the separation device in step c4. Preferably, in step c3 of the process according to the present invention the stream comprising liquid phase contaminant is introduced into a gas-liquid separator, preferably the bottom part of the separation device at a level which is below the level at which the eductor is arranged.

The stream introduced in step c1 has the main function to dilute the slurry and depending on the process conditions to strip some hydrocarbons and/or pre-melt some of the solids in the slurry of contaminants.

Preferably, in the process according to the present in step c4 the stream comprising liquid phase contaminant is removed from the separation device at a level which is below the level at which the eductor is arranged.

Suitably, in step c5 the stream of liquid phase contaminant is removed using a pump.

Preferably, in step c6 the recirculation stream is directly introduced into the eductor.

In the above-described preferred embodiment of step (c), comprising steps c1-c6, a continuously moving slurry phase is obtained, minimizing the risk of any blockages in the cryogenic separation vessel or in the pipelines and other pieces of equipment. Further, when a fully liquid stream is withdrawn from the heat exchanger, the absence of solid contaminant reduces the risk of blockages or erosion in subsequent pipelines or other equipment, and no damages will occur to any devices having moving parts, such as pumps. Moreover, when a pure liquid stream is withdrawn from the heat exchanger, a relatively cold liquid stream is obtained, thus minimizing the heat requirements of the separation device, and maintaining a high amount of exchangeable cold in the product stream.

In the event that the contaminant-rich stream mainly comprises carbon dioxide and is therefore a CO₂-rich stream, preferably CO₂-rich stream is further pressurised and injected into a subterranean formation, preferably for use in enhanced oil recovery or for storage into an aquifer reservoir or for storage into an empty oil reservoir. It is an advantage that a liquid CO₂-rich stream is obtained, as this liquid stream requires less compression equipment to be injected into a subterranean formation.

Gas streams, and in particular natural gas streams produced from a subsurface formation, typically contain water. In order to prevent the formation of gas hydrates in the present process, at least part of the water is suitably removed. Therefore, the gas stream that is used in the present process has preferably been dehydrated. This can be done by conventional processes. A suitable process is the one described in WO-A 2004/070297. Other processes for dehydrating methane or drying natural gas are also possible. Other drying processes include treatment with molecular sieves or drying processes with glycol or methanol. Suitably, water is removed until the amount of water in the natural gas stream comprises at most 50 ppmw, preferably at most 20 ppmw, more preferably at most 1 ppmw of water, based on the total natural gas stream.

The purified hydrocarbon gas that is being recovered after the final separation step can be used as product. The recovered purified hydrocarbon gas may also be subjected to further treatment and/or purification. For instance, the purified hydrocarbon gas may be subjected to fractionation. In the event that the purified hydrocarbon gas is natural gas intended for pipeline transportation or for producing liquefied natural gas (LNG),in order to reach pipeline specifications or LNG specifications the purified natural gas may be further purified. Further purification can for example be done in an additional cryogenic distillation column, suitably with a bottom temperature between −30 and 10° C., preferably between −10 and 5° C. A reboiler may be present to supply heat to the column. Suitably the top temperature column is between −110 and −80° C., preferably between −100 and −90° C. In the top of the cryogenic distillation column a condenser may be present, to provide reflux and a liquefied (LNG) product.

As an alternative, further purification may be accomplished by absorption with a suitable absorption liquid. Suitable absorbing liquids may comprise chemical solvents or physical solvents or mixtures thereof.

A preferred absorbing liquid comprises a chemical solvent and/or a physical solvent, suitably as an aqueous solution.

Suitable chemical solvents are primary, secondary and/or tertiary amines, including sterically hindered amines.

A preferred chemical solvent comprises a secondary or tertiary amine, preferably an amine compound derived from ethanolamine, more especially DIPA, DEA, MMEA (monomethyl-ethanolamine), MDEA (methyldiethanolamine) TEA (triethanolamine), or DEMEA (diethyl-monoethanolamine), preferably DIPA or MDEA. It is believed that these chemical solvents react with acidic compounds such as CO2 and H₂S.

Suitable physical solvents include tetramethylene sulphone (sulpholane) and derivatives, amides of aliphatic carboxylic acids, N-alkyl pyrrolidone, in particular N-methyl pyrrolidine, N-alkyl piperidones, in particular N-methyl piperidone, methanol, ethanol, ethylene glycol, polyethylene glycols, mono- or di(C₁-C₄)alkyl ethers of ethylene glycol or polyethylene glycols, suitably having a molecular weight from 50 to 800, and mixtures thereof. The preferred physical solvent is sulfolane. It is believed that CO2 and/or H₂S are taken up in the physical solvent and thereby removed. Other treatments may include a further compression, when the purified hydrocarbon gas is wanted at a higher pressure. Alternatively, the purified hydrocarbon gas may be subjected to one or more further cooling and separation steps as described above. In this case the purified hydrocarbon gas is subsequently subjected to a total number of combinations of subsequent cooling and separation steps. This number may suitably vary from 2 to 5 combinations.

FIG. 1 shows a flow scheme of an embodiment according to the invention.

In the description of FIG. 1 reference is made to a natural gas stream as an example of a gas stream that may be treated in the process according to the present invention.

A natural gas stream is introduced via a line 1 into a dehydrating unit 21. In the dehydration unit water is being removed from the natural gas stream, e.g., by means of molecular sieves. The water is eventually removed via a line 2. The dehydrated natural gas is passed via a line 3 to a membrane unit 22 where the natural gas stream is separated into a hydrocarbon-rich retentate and a contaminant-rich permeate that is removed via a line 4. As indicated above, the separation may be achieved in more membrane units. As an example only one has been drawn here. The permeate may be compressed in a compressor 23, and passed further via a line 6. Optionally it may be subjected to heat exchange in a unit 28 to condense acidic contaminants. Via a line 7, through a pump 31 and via a line 20 the optionally condensed acidic contaminants are removed from the process.

The hydrocarbon-rich retentate is withdrawn from the membrane unit 22 via a line 5. Line 5 comprises a bundle coil 8 that is located in the lower part of a vessel 24. In the vessel 24 the bundle coil 8 acts as a heat exchanger for solid acidic contaminants that are collected in the bottom of vessel 24. Via line 5, the retentate is passed to a turbo expander 25. From there it is passed via line 9 to a heat exchanger 32. In the turbo-expander 25 and the heat exchanger 32 the retentate is cooled, preferably to a temperature below the dew point of propane. This allows an easy recovery of the propane (and higher hydrocarbons) via a simple gas/liquid separator (not shown). The retentate, optionally without propane and other higher hydrocarbons, leaves the heat exchanger 32 via a line 10, and via an optional further heat exchanger 27 and a further line 11 it is passed to a Joule Thomson valve 29. In the valve the retentate is expanded and thereby cooled to such a temperature that contaminants in the retentate solidify. Line 12 that connects the valve with the vessel 24 is short so that the solids will not block the entry of the retentate to the vessel 24. It is also possible to do away with the line 12 altogether and connect the Joule Thomson valve directly to the wall of vessel 24. In the vessel 24 the solids and liquids, if any, will gather at the bottom of the vessel 24 whereas the vapour, i.e., the purified hydrocarbon gas is removed from the top of the vessel via a line 13.

In the lower part of the vessel 24 the solids are melted by means of the bundle coil 8 through which the relatively warm retentate is passed and which operates as a heat exchanger. The bundle coil is just an example of a way to heat and melt the solid acidic contaminants. Other heating means are also possible. One may use an electric immersion heart, as suggested in WO-A 2007/030888. One may also add relatively warm natural gas liquids to the solid acidic contaminants, as suggested in WO-A 2004/070297. A preferred way is to heat at least part of the liquid that is withdrawn from the vessel 28 via line 16 and recycle thus heated contaminants, which may be liquid or vaporous, into the vessel 24. Combinations of any of these heating means are also possible.

The molten acidic contaminants are withdrawn from the vessel 24 via line 16, and passed to the heat exchanger 32 via a pump 30 and a line 17. In the heat exchanger 32 the liquid contaminants in line 17 and the cold purified hydrocarbon gas in line 13 are indirectly warmed up by the retentate in line 9. The streams are shown in co-current fashion. It is evident to the skilled person that the streams may also be arranged in a counter-current way, e.g., the relatively warm retentate in counter-current with the two other streams. It will be appreciated that it is also feasible to use only one of the other streams or use a stream from another process, such as a stream from an LNG plant and/or an air separation plant. The warmed up liquid acidic contaminants leave the heat exchanger 32 via line 18. The liquid may be combined with the contaminants from membrane unit 22 provided by line 19 and together the acidic contaminants may be removed for further treatment, storage or use in enhanced oil recovery.

The purified hydrocarbon gas leaves heat exchanger 32 via line 14 and after compression in compressor 26 is recovered as product. Dependent on the further use, it may be further purified or transported to where it is to be used. The compression energy for compressor 26 may be provided by expander 25.

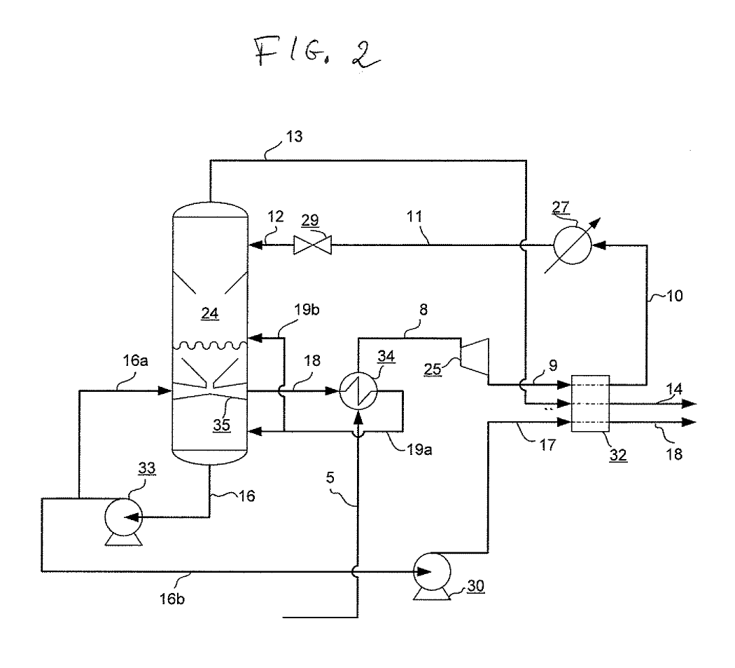

In FIG. 2, a preferred embodiment of step (c) is depicted. The hydrocarbon-rich retentate is withdrawn from the membrane unit 22 (not shown in FIG. 2) via a line 5 and led to a heat exchanger 34. Via line 5, the retentate is passed to a turbo expander 25. From there it is passed via line 9 to a heat exchanger 32. In the heat exchanger 34 solid contaminant present is melted into liquid phase contaminant. Part of this liquid phase contaminant is passed via a conduit 19b as a diluted slurry of contaminants to an intermediate position of separation vessel 24, whereas the main part of liquid phase contaminant is introduced into the bottom part of the separation vessel 24 by means of a conduit 19a. The diluted slurry of contaminants is directed towards the top opening of an eductor 35. In the eductor 35 the diluted slurry is used as a suction fluid and via the eductor 35 it is passed into a heat exchanger 34 via a conduit 18. Liquid phase contaminant is subsequently withdrawn from the separation vessel 24 by means of a conduit 16 using a pump 33. Part of the withdrawn liquid phase contaminant is recovered as a product stream via a conduit 16 and part of said liquid phase contaminant is recycled via a conduit 16a to the eductor 35. As an alternative, pump 33 may also be located in conduit 16a. A funnel (not shown) is present to guide the slurry stream into the direction of conduit 18. Another part of said liquid phase contaminant is led via pump 30 to heat exchanger 32.

The remainder of the retentate is led to turbo expander 25, where it is cooled, preferably to a temperature below the dew point of propane. This allows an easy recovery of the propane (and higher hydrocarbons) via a simple gas/liquid separator (not shown). The retentate, optionally without propane and other higher hydrocarbons, leaves the heat exchanger 32 via a line 10, and via an optional further heat exchanger 27 and a further line 11 it is passed to a Joule Thomson valve 29. In the valve the retentate is expanded and thereby cooled to such a temperature that contaminants in the retentate solidify. Line 12 that connects the valve with the vessel 24 is short so that the solids will not block the entry of the retentate to the vessel 24. It is also possible to do away with the line 12 altogether and connect the Joule Thomson valve directly to the wall of vessel 24. In the vessel 24 the solids and liquids, if any, will gather at the bottom of the vessel 24 whereas the vapour, i.e., the purified hydrocarbon gas is removed from the top of the vessel via a line 13.

Those skilled in the art will appreciate that many modifications and variations are possible in terms of the disclosed embodiments of the invention, configurations, materials and methods without departing from their spirit and scope. Accordingly, the scope of the claims appended hereafter and their functional equivalents should not be limited by particular embodiments described and illustrated herein, as these are merely exemplary in nature.

1. Process for producing purified hydrocarbon gas from a gas stream comprising hydrocarbons and acidic contaminants, which process comprises the steps of:
(a) contacting the gas stream with one or more membranes to obtain a hydrocarbon-rich retentate and a acidic contaminant-rich permeate;
(b) cooling the hydrocarbon-rich retentate in a cooling stage to form a mixture comprising solid and/or liquid acidic contaminants and a vapour comprising vaporous hydrocarbons;
(c) separating the solid and/or liquid acidic contaminants from the mixture, yielding the purified hydrocarbon gas.

2. Process as claimed in claim 1, in which the gas stream has a pressure ranging from 20 to 200 bar.

3. Process as claimed in claim 1, in which the gas stream has a temperature of from −5 to 150° C.

4. Process as claimed in any one of claim 1, in which the gas stream comprises hydrogen sulphide and/or carbon dioxide as acidic contaminants.

5. Process as claimed in claim 4, in which the gas stream comprises from 5 to 40 mol % hydrogen sulphide and from 10 to 90 mol % carbon dioxide, based on the molar amounts of hydrocarbon, hydrogen sulphide and carbon dioxide.

6. Process as claimed in claim 1, in which the membrane comprises a molecular sieve type of membrane, preferably a SAPO-34 membrane.

7. Process as claimed in claim 1, in which the gas stream is contacted with the membrane at a temperature of at least 1° C. above the dew point of the gas stream.

8. Process as claimed in claim 1, in which the cooling stage of the hydrocarbon-rich retentate comprises one or more expansion steps, thereby releasing energy.

9. Process as claimed in claim 8, in which energy that is released by the expansion is used for compressing at least part of the purified hydrocarbon gas.

10. Process as claimed in claim 1 in which the cooling of hydrocarbon-rich retentate includes a step in which the hydrocarbon-rich retentate is cooled to a temperature ranging from 1 to 40° C. above the freeze out temperature of the first acidic contaminant to freeze out, the freeze out temperature being the temperature at which solid contaminants are formed.

11. Process as claimed in claim 1, in which the cooling of hydrocarbon-rich retentate includes a step in which the hydrocarbon-rich retentate is cooled to the temperature at which a mixture of solid and/or liquid acidic contaminants and a vapour comprising vaporous hydrocarbons are formed by expansion over a valve.

12. Process as claimed in claim 1, in which the hydrocarbon retentate is expanded from a pressure ranging from 40 to 200 bar, to a pressure of 10 to 40 bar.

13. Process as claimed in claim 1, in which the gas stream has been dehydrated, preferably resulting in a gas stream comprising less than 50 ppmw of water, based on the total gas stream.

14. Process as claimed in claim 1, wherein step (c) is performed in a separation vessel and comprising the steps of:
(c1) introducing a stream comprising liquid phase acidic contaminants into the intermediate or the bottom part or both of the separation vessel to obtain a diluted slurry of acidic contaminants;
(c2) introducing the diluted slurry of acidic contaminants via an eductor into a heat exchanger in which solid acidic contaminant present in the diluted slurry of contaminants is melted into liquid acidic contaminant, wherein the heat exchanger is positioned outside the separation vessel, and eductor is arranged inside or outside the separation device or partly inside and outside the separation vessel;
(c3) introducing part or all of the liquid contaminant obtained in step c2 into a gas-liquid separator, wherein the gas-liquid separator is preferably the bottom part of the separation vessel;
(c4) introducing part or all of the liquid contaminant obtained in step c3 into the separation vessel;
(c5) removing from the gas-liquid separator a stream of liquid acidic; and
(c6) separating the stream of liquid contaminant obtained in step c5 into a liquid product stream and a recirculation stream which is used as a motive fluid in the eductor.

15. Process according to any claim 1, wherein the purified gas is purified natural gas, the process further comprising the step of cooling the purified natural gas to obtain liquefied natural gas.

16. (canceled)
